(19) United States
(12) Patent Application Publication
(10) Pub. No.: US 2021/0027891 A1
(43) Pub. Date: Jan. 28, 2021

(54) Systems and Methods for Personalized Medication Therapy Management

(71) Applicant: Biosigns Pte. Ltd., Singapore (SG)

(72) Inventors: Kuldeep Singh Rajput, Singapore (SG); Gengbo Chen, Singapore (SG); Maulik D Majmudar, Somerville, MA (US); John Varaklis, Basel (CH); Swaminathan Muthukaruppan, Singapore (SG)

(21) Appl. No.: 17/040,103

(22) PCT Filed: Feb. 26, 2019
(86) PCT No.: PCT/SG2019/050105

§ 371 Date: Sep. 22, 2020

(30) Foreign Application Priority Data
Mar. 23, 2018 (SG) 10201802418S
Aug. 16, 2018 (SG) 10201806932Q

(51) Int. Cl. G16H 50/20; A61B 5/0205; A61B 5/00; G16H 10/60; G16H 50/30

(52) U.S. Cl. CPC G16H 50/20; A61B 5/0205; A61B 5/7267; A61B 5/02416; A61B 5/746; G16H 10/60; G16H 50/30; A61B 5/742

(57) ABSTRACT

A computational therapeutic management system and associate method for providing personalised therapy management for a patient include a therapeutic analytics engine configured to build contextual-specific personalized physiology signature using multivariate data. The personalized physiology signature along with the drug-specific and individual-specific knowledge bases enables the system to evaluate and quantify the therapeutic and adverse effects of drugs on patients. Further, the system monitors patient's health condition, and predicts changes, and provides alarms and reports in a user interface. The clinical annotations by the caregiver/ clinician in the interface are considered as feedback to update the knowledge bases and personalized physiology signature.

### CROSS-REFERENCE TO RELATED APPLICATIONS

This application is the United States national phase of International Application No. PCT/SG2019/050105 filed Feb. 26, 2019, and claims priority to Singapore Patent Application No. 10201802418S filed Mar. 23, 2018, and Singapore Patent Application No. 10201806932Q filed Aug. 16, 2018, the disclosures of which are hereby incorporated by reference in their entirety.

### BACKGROUND OF THE INVENTION

### Field of the Invention

The present disclosure relates to therapeutic management and patient monitoring systems. In a particular form the present disclosure relates to personalised systems to assist clinicians in providing improved therapeutic treatments.

### Description of Related Art

Interventions, both medications and non-medicinal interventions equivalents, such as behaviour modifications, are used to help manage an individual's medical conditions. However, the influence of specific interventions on an individual's physiology can be not only quite variable, but also unpredictable. In certain complex conditions that require combinations of medications, the drug-drug interactions, the drug-disease interactions, and adverse effects can also be serious and unpredictable. Furthermore, some medications and/or interventions need to be closely monitored and/or titrated to find the optimal doses, time and frequency of administering intervention or combinations, and effectiveness durations. In addition to the initiation, application of diagnostic methods, such as blood tests, imaging or other biomarker measurement or the titration of therapeutic agents/interventions, there are specific classes of therapies that are only appropriate if there is an incomplete response to first-line therapies/interventions.

Whilst some therapeutic managements systems exist, they often depend on historical reference database to measure the impact of medication/therapy on patient. As they lack personalization, the ability to identify the subtle changes (either deterioration or improvement) in patient's physiology due to medication/therapy is limited.

There is thus a need to provide a personalised therapeutic management system, or to at least provide a useful alternative to existing systems.

### SUMMARY OF THE INVENTION

According to a first aspect, there is provided a computational therapeutic management system comprising one or more processors and one or more associated memory modules configured to implement:

a data acquisition interface configured to receive, process and store input data, the input data comprising:
physiological data from one or more patient monitoring devices;
contextual data from one or more input devices, the one or more contextual data items relating to actions, locations, activities and/or situational information relating to the patient over a monitoring period;
clinical data on a patient from one or more of an electronic medical record, a digitised caregiver record, a laboratory information management system, and/or a clinical database; a therapeutic analytics engine configured to
generate and update a personalised physiology signature for the patient from the input data, and further configured to use the personalised physiology signature and input data to generate a real-time estimate and/or a daily summary of:
   a Therapeutic Utility Index (TUI), the TUI comprising an estimate of the effectiveness of a medication in meeting a therapy expectation;
   an Adverse Effect Index (AEI) comprising an estimate of the adverse effects of a therapy; and
   a Therapeutic Utility Report (TUR) comprising a summary estimate of the effect of the therapy; and
a therapeutic specific alarm module that generates one or more alarms using the TUI and AEI;

a therapeutic management platform configured to provide a user interface configured to display one or more alarms generated from the TUI and AEI, and the TUR for a patient, and to allow a caregiver/clinician to personalise a therapy for the patient, and to receive annotation data on the TUR from a caregiver/clinician which is processed by the data acquisition interface and the therapeutic analytics engine updates the personalised physiology signature based on the processed annotation data.

In one embodiment, the input data is filtered and pre-processed to exclude poor quality data using a machine learning model trained on annotated poor quality data.

In one embodiment, the input data is segmented to identify one or more time points when there is a change in the contextual data or in the physiological data, and data in a segment is summarised with a start time, an end time, one or more contextual information summaries and one or more summary statistics for physiological data during the segment, and classifying each segment to the personalised physiology signature based on the contextual information.

In one embodiment, the TUI and AEI are obtained by determining a Biovitals Index from the personalised physiology signature, wherein the Biovitals Index has a defined range between a first value and a second value, the first value indicates no change in the patient's condition, and the second value indicates a significant change in the patient's condition, and the TUI and AEI are obtained by measuring one or more deviations of the Biovitals Index and comparing with data stored in a drug specific database comprising information on one or more drugs taken by the patient and a patient specific database, wherein the drug specific database comprises drug-specific information, and the patient specific database comprises data associated with the patient's self-care practices, and disease prognosis extracted from the input data.

In one embodiment, the personalised physiology signature is compared to the segmented data by fitting a vector regression model to obtain a residual vector, wherein the residual vector is used to generate the Biovitals Index, where the first value is 0 and the second value is 1.

In one embodiment, the personalised physiology signature for a patient comprises a personalized database containing physiological data together with contextual data, wherein the contextual data is separated into a plurality of clusters where each cluster corresponds to an ambulatory status of the patient, and the personalized database also stores daily derivatives with the contextual data, and the Biovitals Index is generated by using from the personalised physiology signature as a reference compared with recent input data, and the personalised physiology signature is continuously updated based on new input data.

In one embodiment, the data acquisition interface is further configured to collect patient behaviour data from one or more social media posts, patient reported activities, phone usage information, web browsing history, and eCommerce activity, and wherein the personalised physiology signature is updated based on the received patient behaviour data.

In one embodiment, the one or more patient monitoring devices comprises an ECG and/or PPG sensor, and the therapeutic analytics engine further comprises an ECG and/or PPG analytics module which analyses real time physiological data from the ECG and/or PPG sensor, and integrates the results in the Biovitals Index.

In one embodiment, the input data is used to generate a plurality of clinical daily derivatives, and the TUR is generated by the therapeutic analytics engine from comparing the personalised physiology signature with the plurality of clinical daily derivatives.

In one embodiment, the TUR is generated by the therapeutic analytics engine by applying pattern recognition algorithms and/or applying population-based threshold methods.

According to a second aspect, there is provided a computational method for providing personalised therapy management for a patient comprising:

receiving and processing input data on a patient undergoing a therapy, the input data comprising:
physiological data received from one or more patient monitoring devices;
contextual data received from one or more input devices, the one or more contextual data items relating to actions, locations, activities and/or situational information relating to the patient over a monitoring period; and
clinical data on the patient received from one or more of an electronic medical record, a digitised caregiver record, a laboratory information management system, and/or a clinical database;

generating a personalised physiology signature for the patient from the input data;

generating, using the personalised physiology signature and the input data, one or more real-time estimates and/or a daily summary of:
a Therapeutic Utility Index (TUI), the TUI comprising an estimate of the effectiveness of a medication in meeting a therapy expectation;
an Adverse Effect Index (AEI) comprising an estimate of the adverse effects of a therapy; and
a Therapeutic Utility Report (TUR) comprising a summary estimate of the effect of the therapy; and

processing the TUI and AEI to generate one or more therapeutic specific alarms;

displaying, via a user interface, the one or more therapeutic specific alarms and TUI to a caregiver/clinician;

receiving, via the user interface, changes to a therapy for the patient to personalise the therapy, and/or receive annotation data on the TUR;

updating the personalised physiology signature based on the annotation data if annotation data is received via the user interface.

In one embodiment the method further comprises filtering and pre-processing the input data to exclude poor quality data using a machine learning model trained on annotated poor quality data.

In one embodiment the method further comprises:

segmenting the input data by identifying one or more time points when there is a change in the contextual data or the physiological data, and summarising data in a segment with a start time, an end time, one or more contextual information summaries and one or more summary statistics for physiological data during the segment; and

classifying each segment to the personalised physiology signature based on the contextual information.

In one embodiment the method:

determining a Biovitals Index within a defined range from the personalised physiology signature, wherein the Biovitals Index has a defined range between a first value and a second value, where the first value indicates no change in the patient's condition, and the second value indicates a significant change in the patient's condition; and

generating the TUI and AEI by measuring one or more deviations of the Biovitals Index and comparing with data stored in a drug specific database comprising information on one or more drugs taken by the patient and a patient specific database, wherein the drug specific database comprises drug-specific information, and the patient specific database comprises data associated with the patient's self-care practices, and disease prognosis extracted from the input data.

In one embodiment, determining the Biovitals Index comprises fitting the segmented data to the personalised physiology signature using a vector regression model which generates a residual vector, wherein the residual vector is used to generate the Biovitals Index, where the first value is 0 and the second value is 1.

In one embodiment, the personalised physiology signature for a patient comprises a personalized database containing physiological data together with contextual data, wherein the contextual data is separated into a plurality of clusters where each cluster corresponds to an ambulatory status of the patient, and the personalized database also stores daily derivatives with the contextual data, and the Biovitals Index is generated by using from the personalised physiology signature as a reference compared with recent input data, and the personalised physiology signature is continuously updated based on new input data.

In one embodiment the method further comprises receiving patient behaviour data from one or more social media posts, patient reported activities, phone usage information, web browsing history, and eCommerce activity, and updating the personalised physiology signature is further based on the received patient behaviour data.

In one embodiment, the method further comprises analysing real time physiological data received from an ECG and/or a PPG sensor and integrating the results in the Biovitals Index.

In one embodiment, the method further comprises generating a plurality of clinical daily derivatives from the input data, and generating the TUR by comparing the personalised physiology signature with the plurality of clinical daily derivatives.

In one embodiment, the method further comprises generating the TUR by applying pattern recognition algorithms and/or applying population-based threshold methods.

### BRIEF DESCRIPTION OF THE DRAWINGS

Embodiments of the present disclosure will be discussed with reference to the accompanying drawings wherein:

FIG. 1 is a schematic diagram of the computational therapeutic management system according to an embodiment;

FIG. 2 is a schematic diagram of the therapeutic analytics engine according to an embodiment;

FIG. 3 is a flow chart of a data filtering and pre-processing method according to an embodiment;

FIG. 4 is a flow chart of method of generating a Biovitals Index and updating of the personalised physiology signature according to an embodiment;

FIG. 5 is a schematic diagram of the inputs for updating the personalised physiology signature;

FIG. 6A is an example of the TUI and AEI over a 30 day period for a patient being given a dose of Entresto who does not show adverse effects after an increase in the dose according to an embodiment;

FIG. 6B is an example of the TUI and AEI over a 30 day period for a patient being given a dose of Entresto who does show adverse effects after an increase in the dose according to an embodiment;

FIG. 6C is an example of the TUI and AEI over a 30 day period for a patient being given a dose of Ivabradine who does not show adverse effects over the 30 day period according to an embodiment;

FIG. 6D is an example of the TUI and AEI over a 30 day period for a patient being given a dose of Ivabradine who does develop an adverse effect to the dose over the 30 day period according to an embodiment; and

FIG. 6E is an example of the TUI and AEI over a 300 minute period for a patient being given a dose of Amiodarone who does develop an adverse effect to the dose around 150 minutes after treatment according to an embodiment.

In the following description, like reference characters designate like or corresponding parts throughout the figures.

### DESCRIPTION OF THE INVENTION

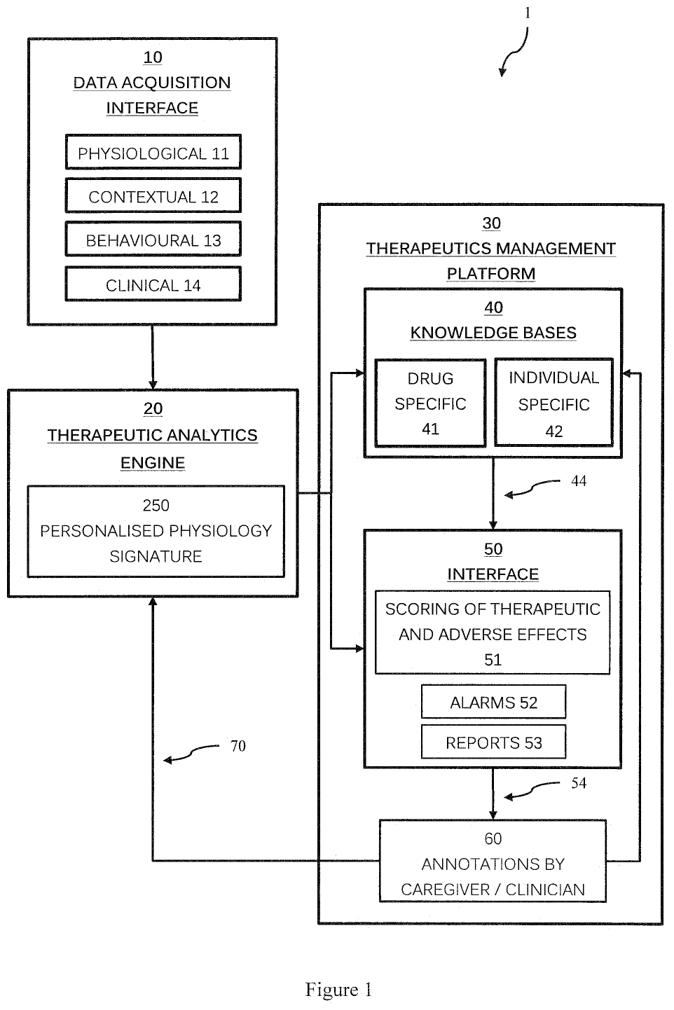

Referring now to FIG. 1, there is shown a computational therapeutic management system 1. The system 1 broadly comprises a data acquisition interface 10 which collects a range of input data including physiological 11, contextual 12, behavioural 13 and clinical 14 data from various sensors, medical devices, the patient, and the caregiver/clinician. A therapeutics analytics engine 20 processes input data and generates and updates a personalised physiology signature for the patient 250, and a therapeutics management platform 30 uses the personalised physiology signature 250, input data from the data acquisition interface 10, and knowledge bases 40, such as drug specific 41 and individual specific 42 databases to generate alarms 52 and reports 53 via a user interface 50. The user interface 50 allows a therapy to be updated and personalised as required, including allowing caregivers/clinicians to provide annotation data on reports and alarms. This can be fed back to the therapeutics analytics engine 20 to update the personalised physiology signature 250.

The system 1 is implemented using a plurality of computational apparatus each including one or more processors and one or more associated memory modules configured to implement the system. The system may be a distributed system including a cloud based system. The therapeutic management system is configured to monitor the effect of a therapy on individuals and to assist the caregiver/clinician to make better and/or personalised therapeutic decisions. The platform can extend from an individual to a group or at an overall population level based on the intended use case. Embodiments of the therapeutic management system will now be described to illustrate the various features and advantages.

The data acquisition interface is configured to receive, process and store input data, such as physiological 11, contextual 12, behavioural 13 and clinical 14 data. The physiological data is received from one or more patient monitoring devices such as wearable sensors/devices and medical equipment, and comprises physiological data such as heart rate, respiration rate, temperature and/or similar data related to physiology. These are captured either continuously (for e.g. heart rate, respiration rate, temperature, ECG, PPG, heart sounds, oxygen saturation) and/or episodically (for e.g. weight or blood pressure measured twice a day) from medical devices, wearable biosensors, ambulatory vital sign monitors, implant devices, manual inputs or any smart portable devices (for e.g. smartphones). Further, the derivatives of the physiology data are also considered as input data, and/or the data data acquisition interface may process raw physiology data to obtain derivatives of the physiology data (which is also considered as input data).

The input data can also comprise of contextual data 12 such as actions, locations, activities, attributes and/or other similar situational information relating to, or associated with the patient over a monitoring period. The contextual data generally reflects the patient's lifestyle and the environment (ie the context of their disease). These are captured either from same devices which capture physiological data (for e.g. activity intensity, step count, position, posture using accelerometer) and/or environment sensors (for e.g. temperature sensor, altitude sensor, air quality sensor) and/or any smart portable devices like smartphones or tablets which capture location, distance, mobile application usage, screen on time, application metadata etc.

In some embodiments input data can also comprise of behavioural data 13 such as interactions from electronic exchanges (call records, email headers, SMS logs), social media posts and interactions, patient reported activities, phone usage information, web browsing history, eCommerce activity, clickstream data, and/or similar information produced as a result of actions by the patient.

Input data can also comprise of clinical data 14 such as administrative and demographic information, diagnosis, treatment, prescription drugs, laboratory test results, clinical notes written by healthcare professional and/or similar data pertaining to the health status of the subject. These are captured either using electronic medical records, a laboratory information management system, a clinical database, a digitised caregiver record or data reported by the subject and/or healthcare professional through questionnaires, surveys, symptoms reporting etc.

The data acquisition interface may be a distributed interface, and may receive data directly from the patient monitoring devices, or indirectly from other components or computing apparatus that receives and/or aggregates physiological data from devices. In some embodiments patients continuously synchronize their physiology biosignals and contextual data from either wearable biosensors, medical devices or implants. Patient reported data and additional contextual data are captured through mobile sensors, a smartphone-based mobile app, or web based user interface. Raw sensor data may be filtered and preprocessed by the data acquisition interface to derive meaningful physiology/context parameters (for e.g. deriving activity intensity, body position, activity classification from 3-axis accelerometer data).

The interface may be provided in a software application running on a portable or local computing apparatus (ie fitbit, wearable devices, smartwatches, smartphones, tablets, laptops, personal desktops) that establishes a connection with a device to download data. This may be a wired connection (eg USB cable), or a wireless connection (e.g. using Bluetooth, Near Field, Wi-Fi, 3G/4G/5G, IEEE 802.11/15, IR, or RF protocols). Alternatively the device may have a local network or internet connection, and may register an address of the data acquisition interface and directly send input data packets to the registered address. In other embodiments the patient monitoring devices may be on a local network (e.g. a hospital network) and the data acquisition interface may execute on a computer forming part of the local network, or the data acquisition interface may establish a connection to such a computer which forwards the data from the devices to the data acquisition interface. Such a local computer may combine data with patient records and device locations to enable linking of data from a device to a patient.

The data acquisition interface 10 provides the input data to a therapeutic analytics engine 20. This is configured to generate and update a personalised physiology signature 250 for the patient from the input data. As will be described the personalised physiology signature and input data is used to generate a real-time estimate and/or a daily summary of a Therapeutic Utility Index (TUI), an Adverse Effect Index (AEI) and a Therapeutic Utility Report (TUR).

The TUI comprising an estimate of the effectiveness of a medication in meeting a therapy expectation. That is, given the expected effects on the physiology, the TUI is a real-time measure of the effectiveness of the medication (therapy) which means how far the therapy meets the expatiation. In one embodiment the TUI varies between 0 and 1 with the greater TUI, the greater positive influence or impact of the therapy.

The AEI comprises an estimate or measure of one or more adverse effects of a therapy, such as a measure of the severity of one or more side effects, or other undesirable outcome. The adverse effect can be known or even unknown and the adverse effects can be measured by but not limited to real-time physiologies, patient reported symptoms, and questionnaire or lab report. As a result, the AEI can be either continuous or episodic. In one embodiment the AEI varies between 0 and 1 with the greater AEI, the worse the adverse effects (e.g. side effects are more severe).

The TUR comprises a summary estimate of the effect of the therapy. This may be generated daily. In some embodiments the TUR can measure the therapy effectiveness if it can be measured by a daily clinical parameter (e.g. hours slept in the case of a sleeping pill).

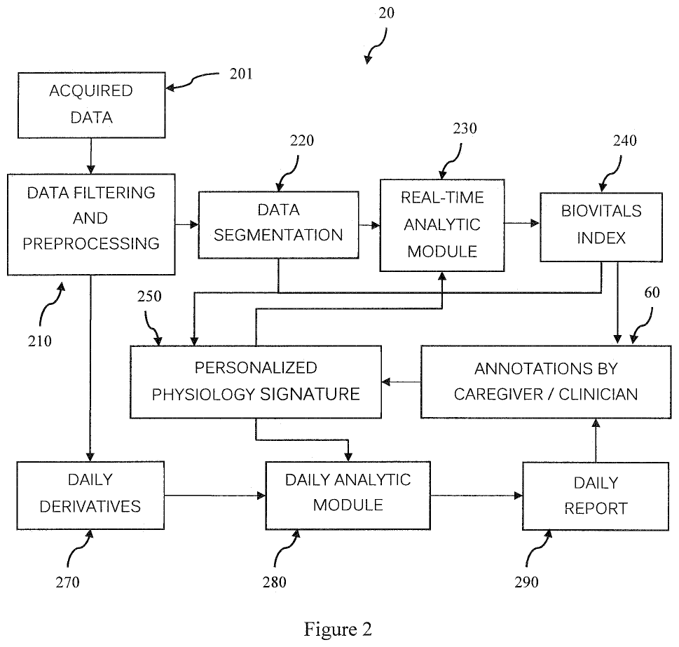

FIG. 2 is a schematic diagram of the therapeutic analytics engine 20 according to an embodiment. In one embodiment the therapeutic analytics engine is a cloud-based data analytic engine implementing various software blocks or modules. In general, it takes the acquired input data, and uses advanced data analysis algorithms to generate meaningful outputs and disease specific alarms for the caregiver to monitor and manage a patient (or patients). At the core of the therapeutic analytics engine is a personalised physiology signature 250 which is generated and updated as additional input data including feedback data is obtained.

In this embodiment the acquired input data 201 (from the data acquisition interface 10) is provided to a data filtering and pre-processing module 210. The cleaned/processed output data is then provided to a data segmentation block 220 to identify time points when changes occur in physiological or contextual data. A real time analytic module 230 processes the segmented data (along with the personalised physiology signature 250) generates a Biovitals Index 240 for a patient from which the TUI and AEI can be obtained. The Biovitals Index 240 is feedback to the personalised physiology signature 250 along with data segmentation data which is provided to a daily analytic module 280. The daily analytic module 280 also receives data from a daily derivatives module 270 which obtains data from the data filtering and pre-processing module 210 to estimate daily estimates of a range of clinical parameters. The daily analytic module 280 generates a daily report (the TUR) which is provided to caregivers/clinicians. The caregivers/clinicians also receive the Biovitals Index 240, and/or the TUI and AEI obtained from the Biovitals Index and can provide annotation data (e.g. via the data acquisition interface 10) which is fed back and used to update the personalised physiology signature 250.

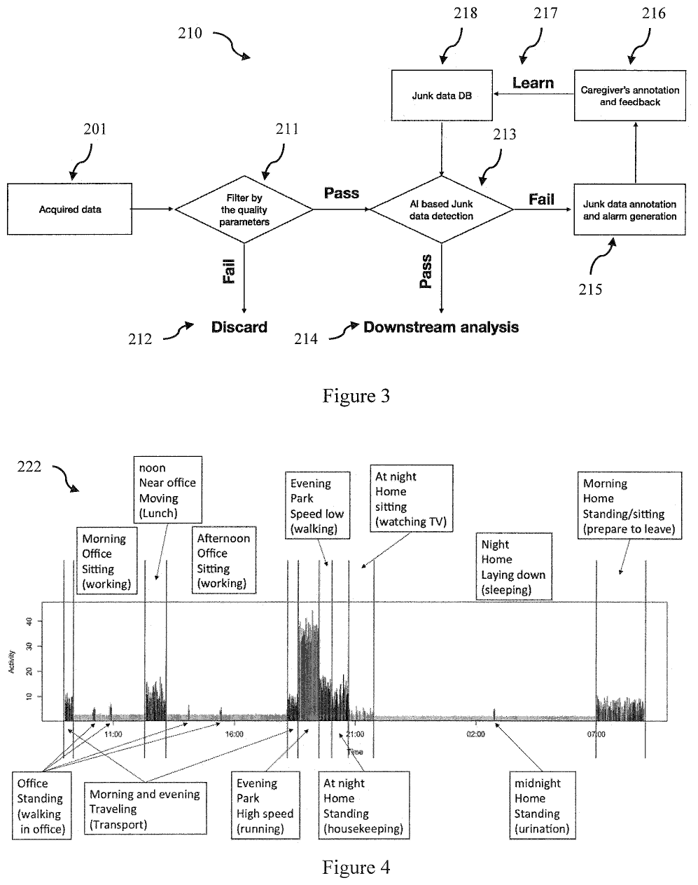

The data filtering and pre-processing module 210 is used to prepare or clean the data for subsequent analysis. FIG. 3 is a flow chart of a data filtering and pre-processing method implemented by the data filtering and pre-processing module 210 according to an embodiment. Ambulatory wearable devices are prone to poor signal quality which affects the performance of the later data analysis algorithms. The poor signal quality can be attributed to many reasons such as improper use of the device, motion artefacts, device malfunctioning. In one embodiment poor-quality data (henceforth referred to as junk data) is detected by filtering the input data using one or more quality parameters provided by the device 211. These quality parameters may be variance estimates, Signal to Noise estimates and other quality metrics based on morphological, statistical and spectral characteristics.

Data which fails the quality filter 211 is discarded 212. Such methods are only effective if the patient is wearing the device properly, which is not always the case. Thus in one embodiment an AI or machine learning based filter is applied 213. A machine learning (ML) classifier is an automated method for assessing the data quality and uses machine/supervised learning methods to build a classifier (or set of classifiers) using reference data sets including test and training sets. In some embodiments deep learning methods using multiple layered classifiers and/or multiple neural nets. In one embodiment a machine learning classifier is a trained artificial neural network that is used to detect the junk data and raise an alarm to the caregiver 215. The caregiver then reviews the flagged junk data and labels or annotates the data, as either acceptable or not 216. Flagged data is added to the junk data database (DB) 218 which is then fed back to the predictive engine 213 to enable learning and so enhance the performance of the machine learning classifier over time. Clean data 214 that passes the junk data detection is then passed onto downstream data processing 214.

In some embodiments data pre-processing is performed to derive meaningful parameters from raw sensor data 201 or clean data 214. For example, speed, location and possible activity type (for e.g. walking, running) can be derived using the GPS sensor data. Data from a 3-axis raw accelerometer can be used to derive the intensity activity and body position of the subject. For example when the patient is standing with no activity, the total acceleration is gravity. When the patient's body position changes the x,y,z accelerometer data will change accordingly. When the patient is doing some activity, the intensity can be reflected in the variation of the accelerometer data. In the engine, algorithms are included to derive the activity intensity and body position from the accelerometer data. In addition, if GPS data is captured, the speed, location (for e.g. home, office, or shopping mall), and possible activity type (for e.g. walking, running or cycling) can also be derived. The data preprocessing is not limited to accelerometer and GPS data. Subject to the data availability the preprocessing also includes the processing of gyroscope meter, light sensor, sound sensor, altitude meter, electric conductance meters and etc.

In one embodiment, pre-processing of input data is performed to obtain sleep stage contextual data. When the patient is sleeping his/her physiology data will change from the day time (for e.g. heart rate and respiration rate will drop), body movement is minimum, and the core temperature will drop. Some clinical parameter during sleep are also critical for the caregiver to monitor the patient (for e.g. the inability lay down properly during sleep and shortness of breath during sleep are critical signs of worsening heart failure). In one embodiment a Hidden Markov Model has been developed to estimate the sleeping stages, and then the sleeping stage is used as one of the contextual parameter in building the personalized physiology signature 250 and generating real time alarms. In this model, the transition between the sleep stages, which is hidden, is a Markov process with transition probabilities. The observed physiology and context data is associated with each sleep stage will different probabilities. The process has been modelled in a Hidden Markov Model and the most likely sleeping stage has been estimated from the context and physiology data.

After the raw data is filtered and pre-processed, data segmentation module 220 uses a data segmentation algorithm to identify the time points when there is change in the contextual data or physiological data. For example, the time points that the patient get up from bed or start to do exercises are identified from the context data. Similarly the time points when the patient's heart rate increases are also identified using the heart rate data. After segmentation, the data within each segment is summarized with start and end time, contextual information and the corresponding summary statistics for physiological data (e.g. means, medians, variance etc of physiological biosignals). Each segment is classified to the personal physiology signature based on the contextual information. By applying the segmentation algorithm, the noise within the segment is significantly reduced and the downstream analysis is more efficient.

FIG. 4 is a schematic illustration of segmentation 222 of input data according to an embodiment. FIG. 4 shows an activity measure (y axis) as a function of time (x axis) over a 24 hour period. The vertical lines indicating the start time and end time of each segment and the boxes indicating the classification. In this example the activity measure is obtained from an accelerometer but in other embodiments it may be obtained from combining multiple data sources.

The segmented physiology data and personal physiology signature is then used by the real-time analytic module 230 to obtain the Biovitals Index 240. In one embodiment segmented physiology data and personal physiology signature is compared using a vector regression model to obtain the residual vectors. In general, the model finds the optimized solution by using the records in personal physiology signature to explain the current physiological data. Then the residual vector, which is the part that cannot be explained, is used to derive the Biovitals Index 240. This index ranges from 0 to 1, where 0 indicates the current physiological data has been observed in the personal physiology signature previously, thus no change in patients' health status (deterioration/improvement). On the other hand, when the index is 1, there is a dramatic change in patients' health status.

In other embodiments, other statistical models or machine learning algorithms may be used to estimate the correlation between the segmented physiology data and the personal physiology signature (ie how the observed data varies from the previous or expected data). In some embodiments the real-time analytic module 230 also includes additional feature detection modules for estimating different parameters which are then integrated into the Biovitals Index (again where 0 means the patient is normal and 1 mean the patient is highly likely to be abnormal). In one embodiment a feature selection module is implemented as a hub and sends different parameters to corresponding analysis algorithms, including AI and machine learning based analysis modules. For example an electrocardiography (ECG) or photoplethysmography (PPG) analytics module which analyses real time physiological data from an ECG or a PPG sensor by performing a rhythm analysis to identify different types of arrhythmia. In some embodiments the algorithms will analyse the ECG data together with the personal physiology signature to filter artefacts and improve the detection accuracy. In some devices, ECG data is not available but the RR interval (inter pulse interval) data can be measured. In one embodiment an algorithm analyses the RR interval sequence in real time and output the risk of atrial fibrillation (AF). Once the risk level exceeds the threshold, a real time alarm is generated, and the caregiver can ask the patient to take a proper ECG and confirm the AF. The algorithm can also learn from the caregiver's annotation and feedback to improve the accuracy.

Many medications are known to have effects on individual's physiology and/or life style. For example, beta-blockers are known to reduce the heart rate for heart failure patients whereas, sleeping pills will increase the sleep time and reduce the daily activity. The effect of the medicine may either impact a person's physiology in real-time, and/or their daily parameters measurements. In the therapeutic management system, one of the key components is the personalised physiology signature (or personalized therapeutic specific model), which in one embodiment includes the medication, dosage, expected outcomes, expect effective duration, known or possible adverse effects etc. Leveraging the known information of the therapies, the clinician/caregiver can personalize the therapy for each patient based on the personalised physiology signature. The personalised physiology signature can also be updated by the clinician/caregiver through the therapeutic management platform when there is a change in the therapy (medicine and/or dosage) or expected positive and/or side effects are updated.

The personal physiology signature 250 is a personalized database containing the subjects' baseline physiological data together with contextual information. Based on the available contextual information, which represents patient's lifestyle in ambulatory setting, the context data is separated into different clusters and each cluster represents one kind of patient's status (for e.g. sleeping, running, sitting in office, intense activity, depression etc.). The personal physiology signature also contains the daily derivatives of the physiological data with the summarized contextual information. The personal physiology signature database is dynamically varying and improving. It is updated when new data collected from the device or patient or caregiver reported inputs.

At the initial stage of the patient monitoring, the personal physiology signature database is empty. The patient monitoring starts by learning the physiological data of the patient and building a database. Based on the availability of the context information, predefined context clusters are then obtained. As data is synchronizing, an algorithm is developed to check that whether the context clusters and the corresponding physiology records are robust and comprehensive enough to make estimation to and generate the Index. Once the initialization process is completed, the algorithms start to generate the Biovitals Index and the personal physiology signature keeps updating.

The Biovitals Index algorithm 230 is a personalized health monitoring model to estimate the health deterioration based on both the context and physiology biosignals in real time. Given the output from the therapeutic analytic engine and the input data, the model will generate an alarm with explanations when the effect of the therapy does not fulfil the expectation or there are severe side effects (or other adverse effects). The alarm together with the explanations will be sent to the therapeutic management platform.

In one embodiment the TUI and AEI are obtained by measuring one or more deviations of the Biovitals Index and comparing with data stored in one or more knowledge bases 40, such as a drug specific data base 41 and a patient specific database 42. The drug specific data base 41 comprises drug-specific information such as pharmacology, pharmacokinetics, indications, contraindications, interactions with other medicines, adverse effects, dosage and administration and/or similar data associated with a drug, for one or more drugs taken by the patient. The patient specific database 42 comprises individual-specific information such as diet compliance, medication adherence, clinical parameters extracted from the physiology data (such as resting heart rate, heart rate recovery etc.) and/or similar data associated with the patient's self-care practices and disease prognosis.

The daily derivatives module 270 processes the acquired data 201 to derive (or obtain) daily estimates (the daily derivatives) of a plurality of important clinical parameters that are known to be significantly related to certain disease. For example, gain in weight (5 lb in 3 days) is significantly related to heart failure. In one embodiment 30 or more daily derivatives are be computed from the acquired data (for e.g. HR recovery, wake up time during sleep, etc.). The daily derivatives are also stored in the personal physiology signature database. The daily derivatives 270 are analysed by the daily analytic module 280 together with the personal physiology signature 250 in order to generate the TUR which comprise a summary estimate of the effect of the therapy. In one embodiment the daily analytic module 280 uses pattern recognition algorithms and/or population-based thresholds methods. The TUR is generated and displayed via a user interface 50 for the caregiver/clinician for review and annotation 60, which is then fed back to the therapeutic analytics engine which triggers updating of the personalised physiology signature 250.

The therapeutic and adverse effects of the drug are quantified by measuring the deviations in the Biovitals Index (if there is any) and/or the daily report. The deviations are then compared with the data stored in drug-specific and individual-specific knowledge bases to obtain the TUI and AEI (e.g. scoring of the therapeutic and adverse effects 51). A therapeutic specific alarm module 52 that generates one or more alarms using the TUI and AEI with explanations when the effect of the therapy does not fulfil the expectation and/or there are severe side or other adverse effects. The alarm together with the explanations will be sent to the therapeutic management platform 30 for display by the interface 50. The user interface 50 is configured to display the alarms, reports, therapeutic utility index and adverse effect index. The caregiver/clinician can use this interface for annotations 60.

The therapeutics management platform 30 provides an interface 50, such as a web application, to allow the caregiver to manage all the patients and alarms. In this platform, the caregiver can view all the alarms, TUI, AEI , and TUR, and take actions, such as communicating with the patient, arranging for a clinic visit, changes in medication or to report false alarms. The caregiver can also raise alarms even the engine did not detect any health deterioration. In the platform both the real time and historical data can be reviewed by the caregiver. The caregiver can annotate the historical data and make comments. The caregiver can also review and update the patient's profile and/or make intervention. The user interface thus allows a clinician to personalise a therapy for the patient.

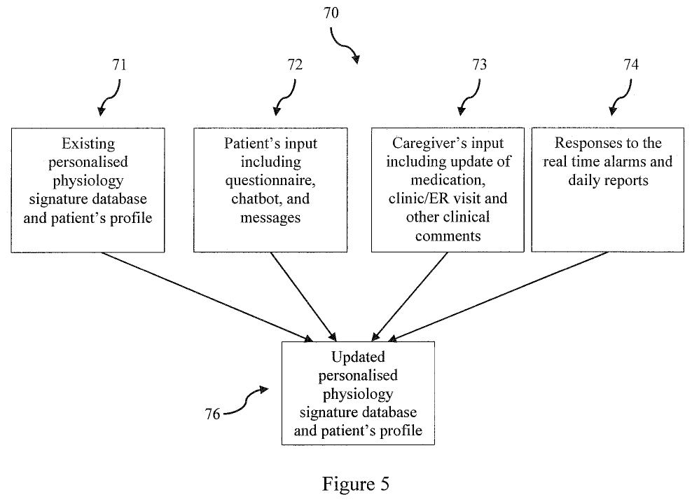

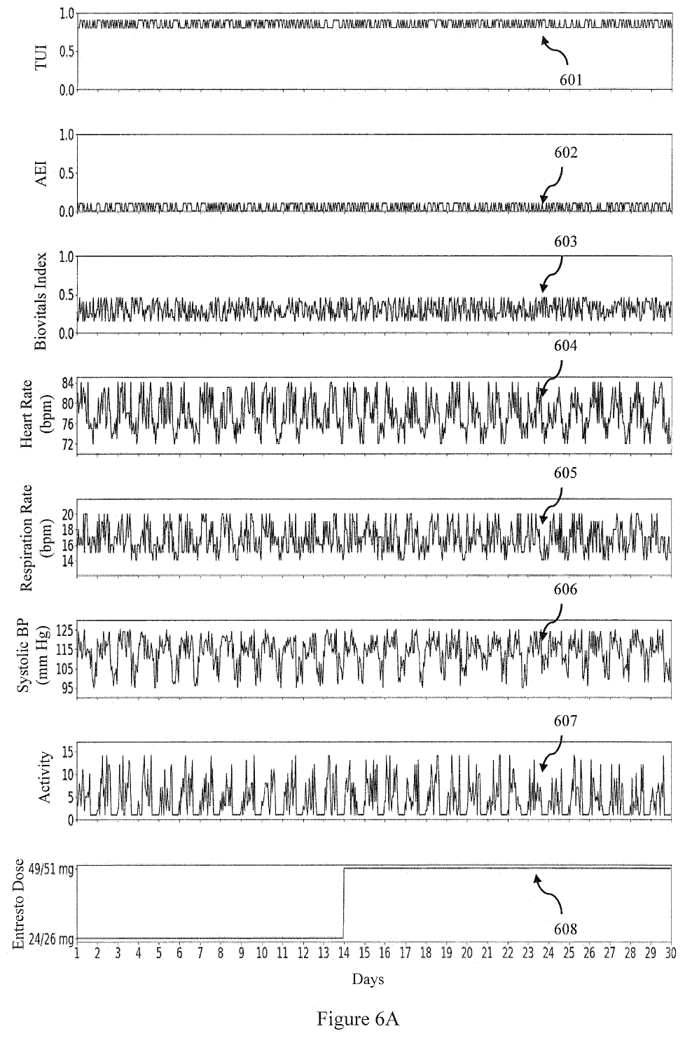

Once new input data is collected or feedback information is received, the engine will trigger the personal physiology signature update module to learn from the new input and update the existing database. This includes processing annotation data obtained via the user interface by the data acquisition interface to update the personalised physiology signature based on the processed annotation data. With this algorithm the personal physiology signature database will be “smarter” as the patients are better learned by the engine over time. FIG. 5 is a schematic diagram of the inputs for updating 70 the personalised physiology signature 250. These include the existing personalised physiology signature database and patient's profile 71; the patient's input including questionnaire, chatbot, and messages 72 (behavioural data 13); the caregiver's input including update of medication, clinic/ER visit and other clinical comments 73 (clinical data 14) and responses to the real time alarms and daily reports 74. This data is combined and used to update the personalised physiology signature 250 database and patient's profile.

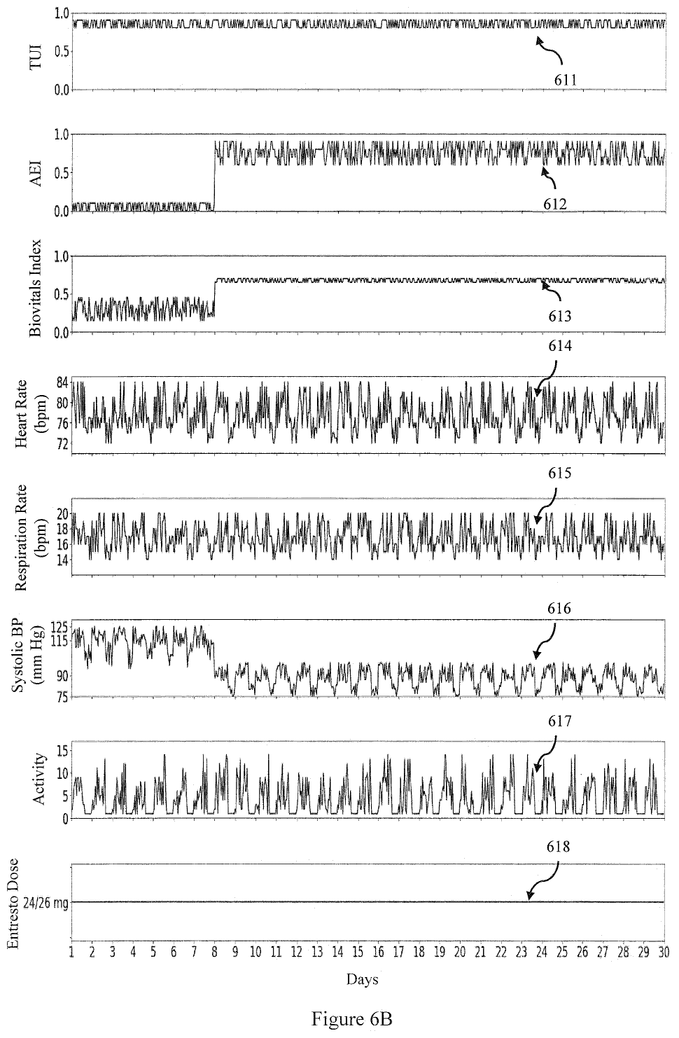

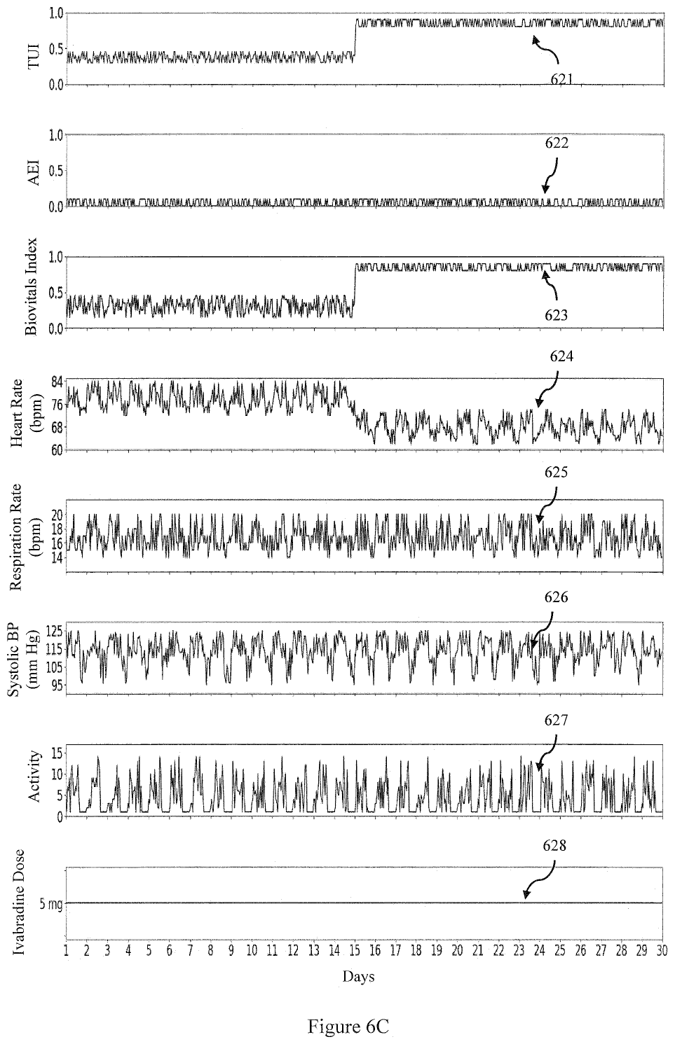

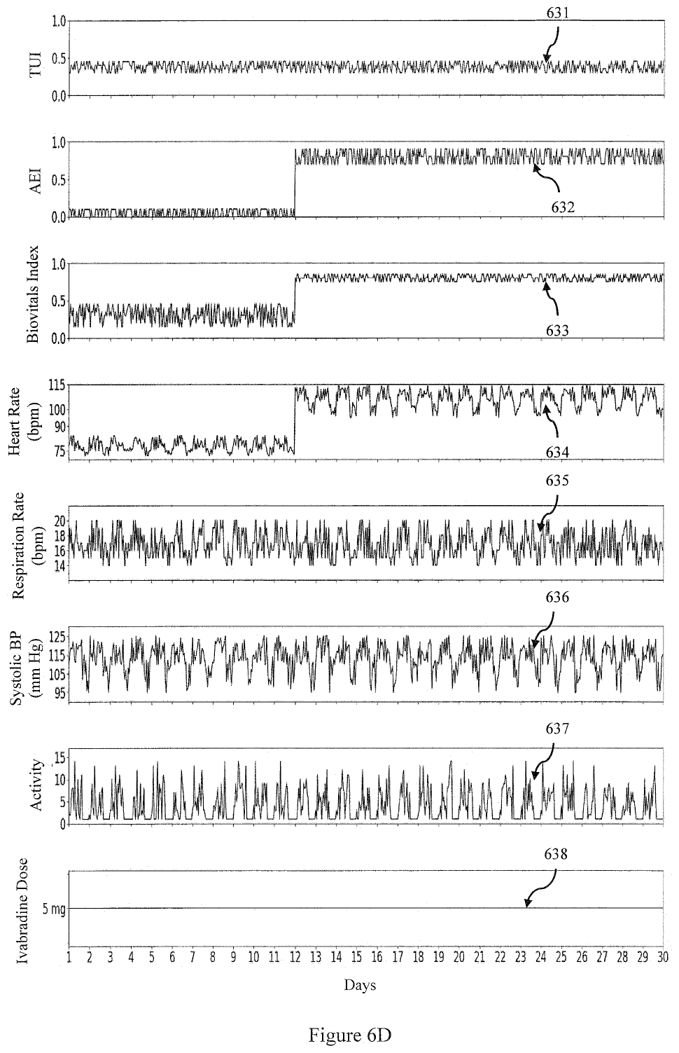

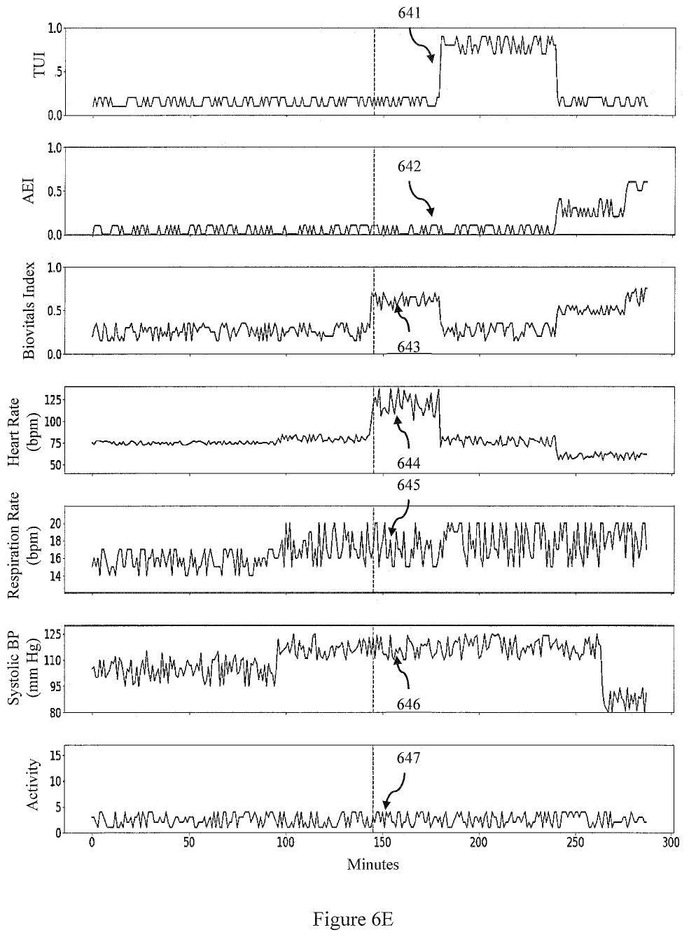

FIGS. 6A to 6E illustrate three examples of the use of an embodiment of the therapeutic management system as described herein. In the first example a therapy using Sacubitril/valsartan is described. Sacubitril/valsartan (Entresto) is the recently approved drug for chronic heart failure (HF) patients with reduced left ventricular ejection fraction (<40%) (rEF) to reduce hospitalization due to HF and death from cardiovascular causes. It has been shown that the patients who are up-titrated to the higher dose and remained on it benefitted the most. The titration guideline is that patients who are tolerated to the lower dose can be up-titrated. In other words, if the patient doesn't present any adverse effects due to the drug, the dose can be up-titrated. The therapeutic effects are improving symptoms (fatigue, shortness of breath), activity and quality of life. The common adverse effects are hypotension, cardiac fibrillation, hyperkalemia, angioedema and renal failure.

Let us consider a scenario when a patient is given Entresto (24/26 mg, twice daily) on Day 1 and the patient doesn't present any adverse effects in the next two weeks. This suggests that the dose can be titrated to 49/51 mg twice daily. FIG. 6A shows plots over a 30 day period of the therapeutic (TUI) 601 adverse effects (AEI) 602, Biovitals Index 603, and physiological data (Heart Rate 604, Respiration rate 605, Systolic blood pressure (BP) 606 and an activity measure 607). As can be seen in this example no adverse effects (e.g. the AEI stays at zero) as the dose is increased 608.

On the other hand, when the patient experiences an adverse effect (for e.g. hypotension, as illustrated by drop in the Systolic BP 615), the adverse effect index 612 and Biovitals Index613 will be high as shown in FIG. 6B.

In a second example, Ivabradine (Corlanor) is indicated to reduce the risk of hospitalization for worsening heart failure with stable, symptomatic chronic heart failure patients with left ventricular ejection fraction <=35%, who are in sinus rhythm with resting heart rate >=70 bpm. The dosage guideline is adjusting the dose to achieve a resting heart rate between 50-60 beats per minute based on tolerability. The therapeutic effect is reducing the resting heart rate. The common adverse effects are bradycardia, hypertension, atrial fibrillation.

Let us consider a scenario when a patient is given Ivabradine (5 mg, twice daily) on Day 1 and the patient's resting heart rate is reduced after two weeks. Both the therapeutic 611 and adverse effects 612 are quantified and shown in FIG. 6C along with the Biovitals Index 613, and physiological data 614, 615, 616, 617). On the other hand, when the patient experiences an adverse effect (for e.g. atrial fibrillation as illustrated by the increase in Heart Rate 634 around day 12), the adverse effect index AEI 632 will be high as shown in FIG. 6D (with a corresponding increase in the Biovitals Index 633).

Amiodarone (Cordarone®) belongs to antidysrhythmic drug class III and has been used to treat severe tachyarrhythmias in both acute and chronic settings. The major adverse effects of this therapy are bradycardia, hypotension, prolonged QT and PR intervals, heart failure and AV blocks. Further, this therapy has significant interactions with other drugs such as beta blockers, warfarin, digoxin, heparin and produces non-desirable effects. Thus, while administering this therapy it is recommended to monitor for lengthening QT interval, bradycardia, and hypotension.

In a third example, a patient is given Amiodarone of initial bolus dose 300 mg in an acute setting to treat arrhythmia. The patient develops both bradycardia and hypotension. The therapeutic and adverse effects are quantified and shown in FIG. 6E. FIG. 6E shows the TUI 641, AEI 642, Biovitals Index 643, Heart Rate 644, Respiration Rate 645, Systolic BP 646 and Activity 647 over a 300 minute period, with the time of the initial bolus does indicated by the dashed line at around 150 minutes. This plot shows that following the development and treatment of arrhythmia (via Amiodarone), at around 180 minutes the Heart Rate 644 is brought down, the Biovitals Index drops and the TUI approaches 1 indicating there is an therapeutic effect of the treatment. However at around 240 minutes, the Heart Rate drops again leading to a first step increase in the Biovitals Index 643 and AEI 642, and a corresponding drop in the TUI (ie the therapeutic effect has reduced/ceased). This is followed by a drop in Systolic BP 646 at around 270 minutes leading to a second step increase in the Biovitals Index 643 and AEI 642, indicating more severe adverse effects of the treatment.

Another example of a clinical scenario which demonstrates the potential value of a therapeutic utility index involves the case of heart failure. Of the many therapeutic agents available for heart failure treatment, the optimal drug of choice as a first-line agent (prioritization of therapies), depends on the type and chronicity of heart failure, along with physiologic response to therapy. The most commonly prescribed classes of medications for heart failure include beta blockers, angiotensin-converting enzyme inhibitors (ACE-I), angiotensin-receptor blockers (ARBs), loop diuretics, aldosterone antagonists, and angiotensin-receptor-neprilysin inhibitors (ARNI), among others. However, most patients cannot tolerate these different agents at once. Furthermore, the titration of therapy to an optimal dose or switching to another therapeutic agent depends heavily on the physiological parameters. In this scenario, the therapeutic management system can help guide the therapeutic decision-making In addition, the longitudinal monitoring of the patient also helps to estimate the potential therapeutic benefit of certain therapeutic agents. Of note, the attached table (Table 1) highlights the starting dose and the optimal dose of the various classes of HF drugs.

For instance, a patient with an acute heart attack resulting in severe ventricular dysfunction may be eligible for several therapies, including anitplatelet agents (e.g. aspirin, clopidogrel), lipid lowering agents (e.g. statins), beta blockers (e.g. carvidelol), ACE-inhibitors (e.g. lisinopril), ARBs (e.g. losartan), aldosterone antagonists (e.g. spironolactone), and loop diuretics (e.g. furosemide). The initial dosing of these medications, along with timing of initiation of these medications, depend on a number of factors, including the hemodynamic state and the physiologic response of the patient to the therapeutic agents. Furthermore, the approach to “up- or down-titration” of these medications to achieve optimal hemodynamic benefit as well as optimal quality of life, may vary widely across clinicians and health systems. Finally, once a patient is optimized on a “stable dose” of many of these medications, and if they suffer an acute decompensation episode of heart failure exacerbation due to volume overload, there needs to be a significant re-adjustment of these medications, as well as introduction of novel therapies, that may perturb the hemodynamic state, as well as the individual contributions and effectiveness of previously prescribed medications. Thus, a real-time, continuous, physiology based remote monitoring system could be incredibly effective in guiding clinical decision making.

This is further illustrated in the following a real world clinical scenario. A 50-year male is diagnosed with new onset heart failure. His heart rate is 100 beats per minute, blood pressure 110/70, respiratory rate 22 breaths per minute, and weight of 175 lbs (about 10 lbs higher than his baseline weight). His blood work reveals sodium 145 mEQ/L, K+4.8 mmol/L, creatinine of 1.9 mg/dL, which is high and suggestive of renal dysfunction. His overall therapeutic management system recommendation will include initiation of several therapies, as the patient is not on any active life-saving therapies, due to his new diagnosis. However, one quickly realizes that his blood pressure is on lower end of normal and any addition of new blood pressure lowering agent could lead to severely low blood pressure, worsening of renal function, and elevation of potassium, all of which can be deadly. Thus, the TUI for an ACE-I will be low, whereas the TUI for beta blocker will be high. In addition, his AEI for ACE-I is going to be high, given the potential for life-threatening complications. Over time, as new medications are initiated, a real-time physiological monitoring system will allow for gradual adjustment of medication doses, as well as initiation or discontinuation of certain medications, to optimize the TUI and minimize the AEI.

TABLE 1
Table 1
Some Drugs for Chronic Heart Failure with Reduced Ejection Fraction¹

| | | | | |
|---|---|---|---|---|
| | Some Oral | Usual Initial | Usual Maximum | |
| Drug | Formulations | Adult Dosage² | Adult Dosage² | Cost³ |

Angiotensin-Converting Enzyme (ACE) inhibitors

| | | | | | | | |
|---|---|---|---|---|---|---|---|
| Captopril* - generic | 12.5, 25, 50, 100 | mg tabs | 6.25 | mg tid | 50 | mg tid | $130.00⁸ |
| Enalapril - generic | 2.5, 5, 10, 20 | mg tabs | 2.5 | mg bid | 20 | mg bid | 12.00⁴ |
| Vasotec (Valeant) | | | | | | | 663.60 |
| Fosinopril⁵ - generic | 10, 20, 40 | mg tabs | 5-10 | mg once/d | 40 | mg once/d | 10.40 |
| Lisinopril - generic | 2.5, 5, 10, 20, 40 | mg tabs | 2.5-5 | mg once/d | 40 | mg once/d | 1.80 |
| Prinivil (Merck) | 5, 10, 20 | mg tabs | | | | | 43.20 |
| Zestril (AstraZeneca) | 2.5, 5, 10, 20, 30, 40 | mg tabs | | | | | 42.30 |
| Perindopril [symbol] - generic | 2, 4, 8 | mg tabs | 2 | mg once/d | 16 | mg once/d | 20.20 |
| Aceon (Symplmed) | | | | | | | 98.10 |
| Quinapril - generic | 5, 10, 20, 40 | mg tabs | 5 | mg bid | 20 | mg bid | 24.10 |
| Accupril (Pfizer) | | | | | | | 171.00 |
| Ramipril - generic | 1.25, 2.5, 5, 10 | mg caps | 1.25-2.5 | mg once/d | 10 | mg once/d | 9.70 |
| Altace (Pfizer) | | | | | | | 129.30 |
| Trandolapril - generic | 1, 2, 4 | mg tabs | 1 | mg once/d | 4 | mg once/d | 17.20 |
| Mavik (Abbvie) | | | | | | | 55.20 |

Angiotensin Receptor Blockers (ARBs)

| | | | | | | | |
|---|---|---|---|---|---|---|---|
| Azilsartan medoxomil* - | 40, 80 | mg tabs | 40-80 | mg once/d | 80 | mg once/d | 135.60 |
| Edarbi (Arbor) | | | | | | | |
| Candesartan cilexetil - | 4, 8, 16, 32 | mg tabs | 4-8 | mg once/d | 32 | mg once/d | 103.10 |
| generic Atacand | | | | | | | 119.40 |
| (AstraZeneca) | | | | | | | |
| Losartan* - generic | 25, 50, 100 | mg tabs | 25-50 | mg once/d | 150 | mg once/d | 5.00 |
| Cozaar (Merck) | | | | | | | 91.00 |
| Valsartan⁵ - generic | 40, 80, 160, 320 | mg tabs | 20-40 | mg bid | 160 | mg bid | 264.40 |
| Dlovan (Novartis) | | | | | | | 277.80 |

Beta-Adrenergic Blockers

| Bisoprolol* - generic | 5, 10 | mg tabs⁶ | 1.25 | mg once/d | 10 | mg once/d | 24.50 |
|---|---|---|---|---|---|---|---|
| Zebeta (Duramed/Barr) | | | | | | | 149.80 |
| Carvedilol - generic | 3.125, 6.25, 12.5, 25 | mg tabs | 3.125 | mg bid | 25 | mg bid | 5.40⁴ |
| Coreg (GSK) | | | | | (50 mg bid for pts >85 kg) | | 172.80 |
| extended-release - | 10, 20, 40, 80 | mg ER caps | 10 | mg once/d | 80 | mg once/d | 173.60 |
| Coreg CR | | | | | | | |
| Metoprolol succinate ER - | | | | | | | |
| generic | 25, 50, 100, 200 | mg ER tabs⁶ | 12.5-25 | mg once/d | 200 | mg once/d | 50.20 |
| Toprol-XL (AstraZeneca) | | | | | | | 85.50 |

Aldosterone Antagonists

| | | | | | | | |
|---|---|---|---|---|---|---|---|
| Eplerenone - generic | 25, 50 | mg tabs | 25 | mg once/d⁶ | 50 | mg once/d⁶ | 104.10 |
| Inspra (Pfizer) | | | | | | | 201.70 |
| Spironolactone [symbol] - | 25, 50, 100 | mg tabs | 12.5-25 | mg once/d⁶ | 25 | mg once/d or bid⁶ | 5.80⁴ |
| generic | | | | | | | |
| Aldactone (Pfizer) | | | | | | | 44.70 |

Vasodilators

| | | | | | |
|---|---|---|---|---|---|
| Isosorbide dinitrate/ | 20/37.5 | mg tabs | 20 mg/37.5 mg tid | 40 mg/ | 228.60 |
| hydralazine^{7 -} | | | | 75 mg tid | |

[symbol]

(Arbor)

[symbol]

Loop Diuretics

| Bumetanide - generic | 0.5, 1.2 | mg tabs | 0.5-1 mg once/d or bid | | 10 mg once/d or | 117.80⁴ |
|---|---|---|---|---|---|---|
| | | | | | in divided doses | |
| Furosemide - generic | 20, 40, 80 | mg tabs | 20-40 mg once/d or bid | | 600 mg once/d or | 192.00⁴ |
| Lasix (Sanofi) | | | | | in divided doses | 288.00 |
| Torsemide - generic | 5,10, 20,100 | mg tabs | 10-20 | mg once/d | 200 mg once/d or | 73.60 |
| Demadex (Meda) | | | | | in divided doses | 487.20 |

Digitalis Glycoside

| | | | | | | | |
|---|---|---|---|---|---|---|---|
| Digoxin - generic | 0.125, 0.25 | mg tabs | 0.125 | mg once/d | 0.125-0.25 | mg once/d | 36.10⁴ |

| | | | | |
|---|---|---|---|---|
| Lanoxin (Covis) | 0.0625, 0.125, 0.1875, 0.25 | mg tabs | or once every other day | 67.80 |

ER = extended-release
*Not approved by the FDA for treatment of heart failure.
¹For treatment of heart failure with reduced ejection fraction (HFrEF).
²Dosage adjustment may be needed for hepatic or renal impairment.
³Approximate WAC for 30 days' treatment at the lowest maximum dosage. WAC = wholesaler acquisition cost or manufacturer's published price to wholesalers; WAC represents a published catalogue or list price and may not represent an actual transactional price. Source: AnalySource ® Monthly. Jan. 5, 2015. Reprinted with permission by First Databank, Inc. All rights reserved. ©2015. www.fdbhealth.com/policies/drug-pricing-policy.
⁴A 30-day supply costs $4.00 at some large discount pharmacies.
⁵Available as scored tablets.
⁶For parents with an eGFR ≥50 mL/min/1.73 m2. For patients with an eGFR 30-49 mL/min/1.73 m2, the initial dose is 25 mg every other day for eplerenone and 12.5 mg once daily or every other day for spironolactone and the maintenance dose is 25 mg once daily for eplerenone and 12.5-25 mg once daily for spironolactone.
⁷Both of these drugs are available generically as single agents, Isosorbide dinitrate is available in 5, 10, 20, and 30-mg tablets and hydralazine in 10, 25, 50, and 100-mg tablets.
⁸FDA-approved as adjunctive therapy for treatment of heart failure in black patients. [symbol]
indicates data missing or illegible when filed

Embodiments of the system are designed to help monitor and manage patients after the patients have taken medication, make interventions, titrate the medications and therefore help the patients to sustain their health status, or homeostasis and ultimately be translated to economic benefit. The therapeutic management system take the data acquired from different resources (physiological parameters from sensors, medication regimen, electronic medical records, etc.) and together with the known positive and negative effects of therapies (on physiological parameters as well as patient-reported symptoms) to estimate a personalised physiology signature. This can then be used to derive the TUI, AEI and TUR as described above. Further updates to the personalised therapy can be made by a clinician/caregiver via the platform (which triggers updates of the personalised physiology signature). Thus the system can evolve with more data from the device, the patients and the caregiver.

Thus in summary, the system continuously monitors the patients, estimates health deterioration and generates both real time alarms and daily reports. Then the caregiver can take action in response to the alarms/reports and make necessary interventions to improve the patient care. The system can thus help to guide the clinician/caregiver in therapeutic decision making and to better manage the patient after the introduction of any new therapies. Consequently, this will improve the prognosis of patients and ultimately be translated to economic benefit.

Those of skill in the art would understand that information and signals may be represented using any of a variety of technologies and techniques. For example, data, instructions, commands, information, signals, bits, symbols, and chips may be referenced throughout the above description may be represented by voltages, currents, electromagnetic waves, magnetic fields or particles, optical fields or particles, or any combination thereof.

Those of skill in the art would further appreciate that the various illustrative logical blocks, modules, circuits, and algorithm steps described in connection with the embodiments disclosed herein may be implemented as electronic hardware, computer software or instructions, middleware, platforms, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The steps of a method or algorithm described in connection with the embodiments disclosed herein may be embodied directly in hardware, in a software module executed by a processor, or in a combination of the two, including cloud based systems. For a hardware implementation, processing may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, or other electronic units designed to perform the functions described herein, or a combination thereof. Various middleware and computing platforms may be used.

In one embodiment a local computing apparatus is used by a clinician or patient which provides an interface to components of the system executing on a remote, web, or cloud based computing apparatus. Additional computing devices, wearables or medical devices are also configured to send data to the remote, web, or cloud based computing apparatus, either directly or via the local computing apparatus. Each computing apparatus comprises at least one processor and a memory operatively connected to the processor, and the computing apparatus is configured to perform the method described herein.

In some embodiments the processor module comprises one or more Central Processing Units (CPUs) configured to perform some of the steps of the methods. A computing apparatus may comprise one or more CPUs. A CPU may comprise an Input/Output Interface, an Arithmetic and Logic Unit (ALU) and a Control Unit and Program Counter element which is in communication with input and output devices through the Input/Output Interface. The Input/Output Interface may comprise a network interface and/or communications module for communicating with an equivalent communications module in another device using a predefined communications protocol (e.g. Bluetooth, Zigbee, IEEE 802.15, IEEE 802.11, TCP/IP, UDP, etc). The computing or terminal apparatus may comprise a single CPU (core) or multiple CPU's (multiple core), or multiple processors. The computing or terminal apparatus may use a parallel processor, a vector processor, or be a distributed computing device, including cloud based computing devices and resources. Memory is operatively coupled to the processor(s) and may comprise RAM and ROM components, and may be provided within or external to the device or processor module. The memory may be used to store an operating system and additional software modules or instructions. The processor(s) may be configured to load and executed the software modules or instructions stored in the memory.

Software modules, also known as computer programs, computer codes, or instructions, may contain a number a number of source code or object code segments or instructions, and may reside in any computer readable medium such as a RAM memory, flash memory, ROM memory, EPROM memory, registers, hard disk, a removable disk, a CD-ROM, a DVD-ROM, a Blu-ray disc, or any other form of computer readable medium. In some aspects the computer-readable media may comprise non-transitory computer-readable media (e.g., tangible media). In addition, for other aspects computer-readable media may comprise transitory computer-readable media (e.g., a signal). Combinations of the above should also be included within the scope of computer-readable media. In another aspect, the computer readable medium may be integral to the processor. The processor and the computer readable medium may reside in an ASIC or related device. The software codes may be stored in a memory unit and the processor may be configured to execute them. The memory unit may be implemented within the processor or external to the processor, in which case it can be communicatively coupled to the processor via various means as is known in the art.

Further, it should be appreciated that modules and/or other appropriate means for performing the methods and techniques described herein can be downloaded and/or otherwise obtained by computing device. For example, such a device can be coupled to a server to facilitate the transfer of means for performing the methods described herein. Alternatively, various methods described herein can be provided via storage means (e.g., RAM, ROM, a physical storage medium such as a compact disc (CD) or floppy disk, etc.), such that a computing device can obtain the various methods upon coupling or providing the storage means to the device. Moreover, any other suitable technique for providing the methods and techniques described herein to a device can be utilized.

Various components of the system may use machine learning (ML) methods, for example for classifying data. These may include machine learning/supervised learning methods to build a classifier (or set of classifiers) using reference data sets including test and training sets, and may include deep learning methods using multiple layered classifiers and/or multiple neural nets. The classifiers may use various signal processing techniques and statistical techniques to identify features, and various algorithms may be used including linear classifiers, regression algorithms, support vector machines, neural networks, Bayesian networks, etc. Various software languages and ML libraries may be used to build the classifier including, TensorFlow, Theano, Torch, PyTorch, Deeplearning4j, Java-ML, scikit-learn, Spark MLlib, Apache MXnet, Azure ML Studio, AML, MATLAB, etc, and the application may be written in high level lanugages such as Python, R, C, C++, C#, Java, etc.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the disclosure is not restricted in its use to the particular application or applications described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope as set forth and defined by the following claims.

1. A computational therapeutic management system comprising one or more processors and one or more associated memory modules configured to implement:
a data acquisition interface configured to receive, process and store input data, the input data comprising:
physiological data from one or more patient monitoring devices;
contextual data from one or more input devices, the one or more contextual data items relating to actions, locations, activities and/or situational information relating to the patient over a monitoring period;
clinical data on a patient from one or more of an electronic medical record, a digitised caregiver record, a laboratory information management system, and/or a clinical database;
a therapeutic analytics engine configured to
generate and update a personalised physiology signature for the patient from the input data, and further configured to use the personalised physiology signature and input data to generate a real-time estimate and/or a daily summary of:
a Therapeutic Utility Index (TUI), the TUI comprising an estimate of the effectiveness of a medication in meeting a therapy expectation;
an Adverse Effect Index (AEI) comprising an estimate of the adverse effects of a therapy; and
a Therapeutic Utility Report (TUR) comprising a summary estimate of the effect of the therapy; and
a therapeutic specific alarm module that generates one or more alarms using the TUI and AEI;
a therapeutic management platform configured to provide a user interface configured to display one or more alarms generated from the TUI and AEI, and the TUR for a patient, and to allow a caregiver/clinician to personalise a therapy for the patient, and to receive annotation data on the TUR from a caregiver/clinician which is processed by the data acquisition interface and the therapeutic analytics engine updates the personalised physiology signature based on the processed annotation data.

2. The system as claimed in claim 1, wherein the input data is filtered and pre-processed to exclude poor quality data using a machine learning model trained on annotated poor quality input data.

3. The system as claimed in claim 1, wherein the input data is segmented to identify one or more time points when there is a change in the contextual data or the physiological data, and data in a segment is summarised with a start time, an end time, one or more contextual information summaries and one or more summary statistics for physiological data during the segment, and classifying each segment to the personalised physiology signature based on the contextual information.

4. The system as claimed in claim 3, wherein the TUI and AEI are obtained by determining a Biovitals Index from the personalised physiology signature, wherein the Biovitals Index has a defined range between a first value and a second value, where the first value indicates no change in the patient's condition, and the second value indicates a significant change in the patient's condition, and the TUI and AEI are obtained by measuring one or more deviations of the Biovitals Index and comparing with data stored in a drug specific database comprising information on one or more drugs taken by the patient and a patient specific database, wherein the drug specific database comprises drug-specific information, and the patient specific database comprises data associated with the patient's self-care practices, and disease prognosis extracted from the input data.

5. The system as claimed in claim 4, wherein the personalised physiology signature is compared to the segmented data by fitting a vector regression model to obtain a residual vector, wherein the residual vector is used to generate the Biovitals Index, where the first value is 0 and the second value is 1.

6. The system as claimed in claim 4, wherein the personalised physiology signature for a patient comprises a personalized database containing physiological data together with contextual data, wherein the contextual data is separated into a plurality of clusters where each cluster corresponds to an ambulatory status of the patient, and the personalized database also stores daily derivatives with the contextual data, and the Biovitals Index is generated by using from the personalised physiology signature as a reference compared with recent input data, and the personalised physiology signature is continuously updated based on new input data.

7. The system as claimed in claim 6, wherein the data acquisition interface is further configured to collect patient behaviour data from one or more social media posts, patient reported activities, phone usage information, web browsing history, and eCommerce activity, and wherein the personalised physiology signature is updated based on the received patient behaviour data.

8. The system as claimed in claim 4, wherein the one or more patient monitoring devices comprises an ECG and/or PPG sensor, and the therapeutic analytics engine further comprises an ECG and/or PPG analytics module which analyses real time physiological data from the ECG and/or PPG sensor, and integrates the results in the Biovitals Index.

9. The system as claimed in claim 1, wherein the input data is used to generate a plurality of clinical daily derivatives, and the TUR is generated by the therapeutic analytics engine from comparing the personalised physiology signature with the plurality of clinical daily derivatives.

10. The system as claimed in claim 9, wherein the TUR is generated by the therapeutic analytics engine by applying pattern recognition algorithms and/or applying population based threshold methods.

11. A computational method for providing personalised therapy management for a patient comprising:
receiving and processing input data on a patient undergoing a therapy, the input data comprising:
physiological data received from one or more patient monitoring devices;
contextual data received from one or more input devices, the one or more contextual data items relating to actions, locations, activities and/or situational information relating to the patient over a monitoring period; and
clinical data on the patient received from one or more of an electronic medical record, a digitised caregiver record, a laboratory information management system, and/or a clinical database;
generating a personalised physiology signature for the patient from the input data;
generating, using the personalised physiology signature and the input data, one or more real-time estimates and/or a daily summary of:
a Therapeutic Utility Index (TUI), the TUI comprising an estimate of the effectiveness of a medication in meeting a therapy expectation;
an Adverse Effect Index (AEI) comprising an estimate of one or more adverse effects of a therapy; and
a Therapeutic Utility Report (TUR) comprising a summary estimate of the effect of the therapy; and
processing the TUI and AEI to generate one or more therapeutic specific alarms;
displaying, via a user interface, the one or more therapeutic specific alarms and TUI to a caregiver/clinician;
receiving, via the user interface, changes to a therapy for the patient to personalise the therapy, and/or receive annotation data on the TUR;
updating the personalised physiology signature based on the annotation data if annotation data is recieved via the user interface.

12. The method as claimed in claim 11, further comprising filtering and pre-processing the input data to exclude poor quality data using a machine learning model trained on annotated poor quality data.

13. The method as claimed in claim 11, further comprising:
segmenting the input data by identifying one or more time points when there is a change in the contextual data or the physiological data, and summarising data in a segment with a start time, an end time, one or more contextual information summaries and one or more summary statistics for physiological data during the segment; and
classifying each segment to the personalised physiology signature based on the contextual information.

14. The method as claimed in claim further comprising:
determining a Biovitals Index within a defined range from the personalised physiology signature, wherein the Biovitals Index has a defined range between a first value and a second value, where the first value indicates no change in the patient's condition, and the second value indicates a significant change in the patient's condition; and
generating the TUI and AEI by measuring one or more deviations of the Biovitals Index and comparing with data stored in a drug specific database comprising information on one or more drugs taken by the patient and a patient specific database, wherein the drug specific database comprises drug-specific information, and the patient specific database comprises data associated with the patient's self-care practices, and disease prognosis extracted from the input data.

15. The method as claimed in claim 14, wherein determining the Biovitals Index comprises fitting the segmented data to the personalised physiology signature using a vector regression model which generates a residual vector, wherein the residual vector is used to generate the Biovitals Index, where the first value is 0 and the second value is 1.

16. The method as claimed in claim 14, wherein the personalised physiology signature for a patient comprises a personalized database containing physiological data together with contextual data, wherein the contextual data is separated into a plurality of clusters where each cluster corresponds to an ambulatory status of the patient, and the personalized database also stores derivatives with the contextual data, and the Biovitals Index is generated by using from the personalised physiology signature as a reference compared with recent input data, and the personalised physiology signature is continuously updated based on new input data.

17. The method as claimed in claim 11, further comprising receiving patient behaviour data from one or more social media posts, patient reported activities, phone usage information, web browsing history, and eCommerce activity, and updating the personalised physiology signature is further based on the received patient behaviour data.

18. The method as claimed in claim 14, further comprising analysing real time physiological data received from an ECG and/or a PPG sensor and integrating the results in the Biovitals Index.

19. The method as claimed in claim 11, further comprising generating a plurality of clinical daily derivatives from the input data, and generating the TUR by comparing the personalised physiology signature with the plurality of clinical daily derivatives.

20. The method as claimed in claim 19, wherein the TUR is generated by applying pattern recognition algorithms and/or applying population-based threshold methods.
